(19) United States
(12) Patent Application Publication
(10) Pub. No.: US 2009/0291973 A1
(43) Pub. Date: Nov. 26, 2009

(54) Novel Quinazoline Derivatives and Their Medical Use

(75) Inventors: William Dalby Brown, Soborg (DK); Lene Teuber, Vaelose (DK); Tino Dyhring, Solrod (DK); Dorte Strøbæk, Farum (DK); Carsten Jessen, Birkerod (DK)

Correspondence Address: BIRCH STEWART KOLASCH & BIRCH, PO BOX 747, FALLS CHURCH, VA, 22040-0747, US

(73) Assignee: NeuroSearch A/S, Ballerup (DK)

(21) Appl. No.: 12/085,188

(22) PCT Filed: Nov. 17, 2006
(86) PCT No.: PCT/EP2006/068627

§ 371 Date: Oct. 16, 2008

Related U.S. Application Data
(60) Provisional application No. 60/738,052, filed on Nov. 21, 2005.

(30) Foreign Application Priority Data
Nov. 18, 2005 (DK) PA 2005 01615

(51) Int. Cl. A61K 31/517; C07D 239/72

(52) U.S. Cl. 514/266.3; 544/287

(57) ABSTRACT

This invention relates to novel quinazoline derivatives having medical utility, to use of the quinazoline derivatives of the invention for the manufacture of a medicament, to pharmaceutical compositions comprising the quinazoline derivatives of the invention, and to methods of treating a disorder, disease or a condition of a subject, which disorder, disease or condition is responsive to activation of Kᵥ7 channels.

### TECHNICAL FIELD

This invention relates to novel quinazoline derivatives having medical utility, to use of the quinazoline derivatives of the invention for the manufacture of a medicament, to pharmaceutical compositions comprising the quinazoline derivatives of the invention, and to methods of treating a disorder, disease or a condition of a subject, which disorder, disease or condition is responsive to activation of Kᵥ7 channels.

### BACKGROUND ART

Potassium (K⁺) channels are structurally and functionally diverse families of K⁺-selective channel proteins, which are ubiquitous in cells, indicating their central importance in regulating a number of key cell functions. While widely distributed as a class, K⁺ channels are differentially distributed as individual members of this class or as families.

Recently a new family of potassium channels, the KCNQ channels, has attracted attention as target for therapeutic development. The human KCNQ1 channel was disclosed by Wang, Q et al. [Wang, Q et al.; Nature Genet. 1996 12 17-23], the human KCNQ2 channel was disclosed by Biervert et al. [Biervert et al.; Science 1998 279 403-406]; the human KCNQ3 channel was disclosed by Schroeder et al. [Schroeder et al.; Nature 1998 396 687-690]; the human KCNQ4 channel was disclosed by Kubisch et al. [Kubisch et al.; Cell 1999 96 (3) 437-46]; and the human KCNQ5 channel was disclosed by Schroeder et al. [Schroeder et al.; J. Biol. Chem. 2000 275 (31) 24089-24095]. According to the latest nomenclature KCNQ1-KCNQ5 channels now are also designated Kᵥ7.1-Kᵥ7.5.

Due to the distribution of KCNQ channels within the organism, KCNQ channel modulators are considered potentially useful for the treatment or alleviation of conditions as diverse as pain, migraine, tension type headache, CNS disorders, CNS damage caused by trauma, stroke or neurodegenerative illness or diseases, learning and cognitive disorders, motion and motor disorders, multiple sclerosis, heart failure, cardiomyopathia, cardiac disorders, inflammatory diseases, ophthalmic conditions, progressive hearing loss or tinnitus, obstructive or inflammatory airway diseases, for inducing or maintaining bladder control including the treatment or prevention of urinary incontinence.

WO 2005025293 discloses fused ring heterocycles useful as potassium channel modulators. However, the quinazoline derivatives of the present invention are not described.

### SUMMARY OF THE INVENTION

It is an object of the present invention to provide novel quinazoline derivatives having medical utility for combating disorders, diseases or conditions responsive to activation of Kᵥ7 channels.

In its first aspect the invention provides quinazoline derivatives of Formula I

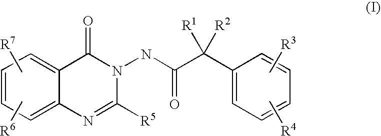

any of its enantiomers or any mixture of its enantiomers, or a pharmaceutically-acceptable addition salt thereof, or an N-oxide thereof, wherein

R¹ represents hydrogen or alkyl; and

R² represents alkyl, cycloalkyl, halo, haloalkyl, hydroxyalkyl, hydroxy, alkoxy, phenyl, phenylalkyl, amino, alkyl-carbonyl-amino, cyano or nitro; or

R¹ and R² together with the carbon atom to which they are attached form a cycloalkyl group; or

R¹ represents hydrogen; and R² together with R³ attached in ortho-position on the aromatic ring form a —(CH₂)ₙ— bridge, wherein n is 1, 2 or 3;

R³ and R⁴, independently of each other, represent hydrogen, alkyl, cycloalkyl, halo, haloalkyl, hydroxy, alkoxy, haloalkoxy, amino, alkyl-carbonyl-amino, alkyl-sulfonyl, phenyl, benzoyl, cyano or nitro; or

R³ and R⁴ together form a methylenedioxy group; or

R³ attached in ortho-position on the aromatic ring and together with R² form a —(CH₂)ₙ— bridge, wherein n is 1, 2 or 3; and R⁴ is as defined above;

R⁵ represents alkyl, cycloalkyl, alkoxy, alkylthio or phenyl; and

R⁶ and R⁷, independently of each other, represent hydrogen, alkyl, cycloalkyl, halo, haloalkyl, hydroxy, alkoxy, haloalkoxy, amino, alkyl-carbonyl-amino (acetamido), nitro, cyano or phenyl.

In another aspect the invention provides pharmaceutical compositions comprising a therapeutically effective amount of the quinazoline derivative of the invention, or a pharmaceutically-acceptable addition salt thereof.

Viewed from a third aspect the invention relates to the use of the quinazoline derivative of the invention, or a pharmaceutically-acceptable addition salt thereof, for the manufacture of pharmaceutical compositions.

In a fourth aspects the invention provides a method of treatment, prevention or alleviation of a disease or a disorder or a condition of a living animal body, including a human, which disorder, disease or condition is responsive to activation of Kᵥ7 channels, which method comprises the step of administering to such a living animal body in need thereof, a therapeutically effective amount of the quinazoline derivative of the invention, or a pharmaceutically-acceptable addition salt thereof.

Other objects of the invention will be apparent to the person skilled in the art from the following detailed description and examples.

### DETAILED DISCLOSURE OF THE INVENTION

The quinazoline derivatives of the invention may be characterised by Formula

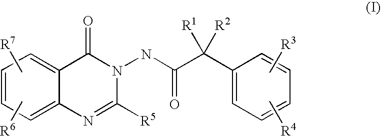

any of its enantiomers or any mixture of its enantiomers, or a pharmaceutically-acceptable addition salt thereof, or an N-oxide thereof, wherein

R¹ represents hydrogen or alkyl; and

R² represents alkyl, cycloalkyl, halo, haloalkyl, hydroxyalkyl, hydroxy, alkoxy, phenyl, phenylalkyl, amino, alkyl-carbonyl-amino, cyano or nitro; or

R¹ and R² together with the carbon atom to which they are attached form a cycloalkyl group; or

R¹ represents hydrogen; and R² together with R³ attached in ortho-position on the aromatic ring form a —(CH₂)ₙ— bridge, wherein n is 1, 2 or 3;

R³ and R⁴, independently of each other, represent hydrogen, alkyl, cycloalkyl, halo, haloalkyl, hydroxy, alkoxy, haloalkoxy, amino, alkyl-carbonyl-amino, alkyl-sulfonyl, phenyl, benzoyl, cyano or nitro; or

R³ and R⁴ together form a methylenedioxy group; or

R³ attached in ortho-position on the aromatic ring and together with R² form a —(CH₂)ₙ— bridge, wherein n is 1, 2 or 3; and R⁴ is as defined above;

R⁵ represents alkyl, cycloalkyl, alkoxy, alkylthio or phenyl; and

R⁶ and R⁷, independently of each other, represent hydrogen, alkyl, cycloalkyl, halo, haloalkyl, hydroxy, alkoxy, haloalkoxy, amino, alkyl-carbonyl-amino (acetamido), nitro, cyano or phenyl;

provided, however, that if R¹ is hydrogen, R² is methyl, R³ and R⁴ represent hydrogen, R⁵ is isopropyl, and R⁶ and R⁷ represent hydrogen, then the compound it is not a quinazoline derivative racemate but the R- or S-enantiomer of the quinazoline derivative.

In a preferred embodiment the quinazoline derivative of the invention is a compound of Formula I, wherein R¹ represents hydrogen or alkyl.

In a more preferred embodiment R¹ represents hydrogen.

In another more preferred embodiment R¹ represents alkyl.

In a still more preferred embodiment R¹ represents methyl.

In another preferred embodiment the quinazoline derivative of the invention is a compound of Formula I, wherein

In a more preferred embodiment R² represents alkyl, cycloalkyl, halo, haloalkyl, hydroxyalkyl, hydroxy, alkoxy, phenyl, phenylalkyl, amino, alkyl-carbonyl-amino, cyano or nitro.

In an even more preferred embodiment R² represents alkyl, cycloalkyl, halo, hydroxyalkyl, hydroxy, phenyl, benzyl, amino, alkyl-carbonyl-amino or cyano.

In a still more preferred embodiment R² represents methyl, ethyl, isopropyl, 2-butyl, cyclopentyl, cyclohexyl, fluoro, hydroxymethyl, hydroxy, phenyl, benzyl, amino, isobutyl-carbonyl-amino or cyano.

In a yet more preferred embodiment R² represents methyl, ethyl, isopropyl, cyclohexyl, fluoro or benzyl.

In a further more preferred embodiment R² represents alkyl.

In a yet further more preferred embodiment R² represents methyl, ethyl, isopropyl or 2-butyl.

In a yet further more preferred embodiment R² represents methyl, ethyl or isopropyl.

In a third preferred embodiment the quinazoline derivative of the invention is a compound of Formula I, wherein R¹ represents hydrogen or methyl; and R² represents methyl, ethyl, isopropyl, cyclohexyl, fluoro or benzyl.

In a more preferred embodiment R¹ represents hydrogen or alkyl; and R² represents alkyl.

In an even more preferred embodiment R¹ represents alkyl; and R² represents alkyl.

In a still more preferred embodiment R¹ represents methyl; and R² represents methyl, ethyl, isopropyl or 2-butyl.

In a fourth preferred embodiment the quinazoline derivative of the invention is a compound of Formula I, wherein R¹ and R² together with the carbon atom to which they are attached form a cycloalkyl group.

In a more preferred embodiment R¹ and R² together with the carbon atom to which they are attached form a cyclopropyl group.

In a fifth preferred embodiment the quinazoline derivative of the invention is a compound of Formula I, wherein R¹ represents hydrogen; and R² together with R³ attached in ortho-position on the aromatic ring form a —(CH₂)ₙ— bridge, wherein n is 1, 2 or 3.

In a more preferred embodiment R¹ represents hydrogen; and R² together with R³ attached in ortho-position on the aromatic ring form a —(CH₂)— bridge.

In a sixth preferred embodiment the quinazoline derivative of the invention is a compound of Formula I, wherein R³ and R⁴, independently of each other, represent hydrogen, alkyl, cycloalkyl, halo, haloalkyl, hydroxy, alkoxy, haloalkoxy, amino, alkyl-carbonyl-amino, alkyl-sulfonyl, phenyl, benzoyl, cyano or nitro; or R³ and R⁴ together form a methylenedioxy group.

In a more preferred embodiment R³ and R⁴, independently of each other, represent hydrogen, alkyl, halo, haloalkyl, alkoxy or benzoyl.

In an even more preferred embodiment R³ and R⁴, independently of each other, represent hydrogen, methyl, isopropyl, isobutyl, fluoro, chloro, bromo, trifluoromethyl, methoxy or benzoyl.

In a still more preferred embodiment R³ and R⁴, independently of each other, represent hydrogen or halo.

In a yet more preferred embodiment R³ represents hydrogen, or halo; and R⁴ represents alkyl, cycloalkyl, halo, haloalkyl, hydroxy, alkoxy, haloalkoxy, amino, alkyl-carbonyl-amino, alkyl-sulfonyl, phenyl, benzoyl, cyano or nitro.

In a further more preferred embodiment R³ represents hydrogen or halo; and R⁴ represents alkyl, halo, haloalkyl, alkoxy or benzoyl.

In a yet further more preferred embodiment R³ represents hydrogen or fluoro; and R⁴ represents methyl, isopropyl, isobutyl, fluoro, chloro, bromo, trifluoromethyl, methoxy or benzoyl.

In a yet further more preferred embodiment R³ represents hydrogen; and R⁴ represents halo.

In a yet further more preferred embodiment R³ represents hydrogen; and R⁴ represents fluoro, chloro or bromo.

In a yet further more preferred embodiment R³ and R⁴ both represent hydrogen.

In a yet further more preferred embodiment R³ and R⁴ together form a methylenedioxy group.

In a seventh preferred embodiment the quinazoline derivative of the invention is a compound of Formula I, wherein R³ attached in ortho-position on the aromatic ring and together with R² form a —(CH₂)ₙ— bridge, wherein n is 1, 2 or 3; and R⁴ is as defined above.

In a more preferred embodiment R³ attached in ortho-position on the aromatic ring and together with R² form a —(CH₂)— bridge; and R⁴ is hydrogen.

In an eight preferred embodiment the quinazoline derivative of the invention is a compound of Formula I, wherein R⁵ represents alkyl, cycloalkyl, alkoxy, alkylthio or phenyl.

In a more preferred embodiment R⁵ represents methyl, ethyl, isopropyl, 2-butyl, cyclopropyl, cyclohexyl, methoxy, ethoxy, methylsulfanyl, ethylsulfanyl, isopropylsulfanyl or phenyl.

In an even more preferred embodiment R⁵ represents methyl, ethyl, isopropyl, 2-butyl, cyclopropyl, cyclohexyl, isopropylsulfanyl or phenyl.

In a still more preferred embodiment R⁵ represents alkyl or alkylthio.

In a yet more preferred embodiment R⁵ represents isopropyl or isopropylsulfanyl.

In a ninth preferred embodiment the quinazoline derivative of the invention is a compound of Formula I, wherein R⁶ and R⁷, independently of each other, represent hydrogen, alkyl, cycloalkyl, halo, haloalkyl, hydroxy, alkoxy, haloalkoxy, amino, alkyl-carbonyl-amino (acetamido), nitro, cyano or phenyl.

In a more preferred embodiment R⁶ and R⁷, independently of each other, represent hydrogen, methyl, fluoro, chloro, bromo, trifluoromethyl, hydroxy, methoxy, ethoxy, amino, or acetamido or cyano.

In an even more preferred embodiment R⁶ and R⁷, independently of each other, represent hydrogen, methyl, fluoro, chloro, bromo, trifluoromethyl, hydroxy, methoxy, amino or acetamido.

In a still more preferred embodiment R⁶ represents hydrogen, trifluoromethyl; and R⁷ represents hydrogen, methyl, fluoro, chloro, bromo, trifluoromethyl, hydroxy, methoxy, ethoxy, amino, acetamido or cyano.

In a yet more preferred embodiment R⁶ and R⁷, independently of each other, represent hydrogen, halo or haloalkyl.

In a yet further more preferred embodiment R⁶ and R⁷, independently of each other, represent hydrogen, halo, haloalkyl or cyano.

In a yet further more preferred embodiment R⁶ and R⁷, independently of each other, represent hydrogen, fluoro, chloro, bromo, trifluoromethyl or cyano.

In a yet further more preferred embodiment R⁵ represents hydrogen; and R⁷ represents hydrogen, halo or haloalkyl.

In a yet further more preferred embodiment R⁶ represents hydrogen; and R⁷ represents hydrogen, chloro or trifluoromethyl.

In a most preferred embodiment the quinazoline derivative of the invention is
N-(2-Isopropyl-4-oxo-4H-quinazolin-3-yl)-3-methyl-2-phenyl-butyramide;
2-(4-Chloro-phenyl)-N-(2-isopropylsulfanyl-4-oxo-4H-quinazolin-3-yl)-butyramide;
2-(3,5-Difluoro-phenyl)-N-(2-isopropylsulfanyl-4-oxo-7-trifluoromethyl-4H-quinazolin-3-yl)-propionamide;
N-(2-Ethyl-7-fluoro-4-oxo-4H-quinazolin-3-yl)-2-phenyl-propionamide;
(S)-N-(2-Isopropyl-4-oxo-4H-quinazolin-3-yl)-2-phenyl-propionamide;
N-(2-Isopropyl-4-oxo-4H-quinazolin-3-yl)-2,3-diphenyl-propionamide;
Bicyclo[4.2.0]octa-1,3,5-triene-7-carboxylic acid (2-isopropyl-4-oxo-4H-quinazolin-3-yl)-amide;
N-(2-Isopropyl-4-oxo-4H-quinazolin-3-yl)-2-p-tolyl-propionamide;
2-Cyclohexyl-N-(2-isopropyl-4-oxo-4H-quinazolin-3-yl)-2-phenyl-acetamide;
2-(3-Benzoyl-phenyl)-N-(2-isopropyl-4-oxo-4H-quinazolin-3-yl)-propionamide;
1-Phenyl-cyclopropanecarboxylic acid (2-isopropyl-4-oxo-4H-quinazolin-3-yl)-amide;
2-(3,4-Dimethoxy-phenyl)-N-(2-isopropyl-4-oxo-4H-quinazolin-3-yl)-propionamide;
(R)-N-(2-Isopropyl-4-oxo-4H-quinazolin-3-yl)-2-phenyl-propionamide;
N-(2-Isopropyl-4-oxo-4H-quinazolin-3-yl)-2-phenyl-isobutyramide;
2-(4-Chloro-phenyl)-N-(2-isopropyl-4-oxo-7-trifluoromethyl-4H-quinazolin-3-yl)-propionamide;
N-(2-Isopropyl-4-oxo-4H-quinazolin-3-yl)-2-(4-trifluoromethyl-phenyl)-propionamide;
2-(3,4-Dichloro-phenyl)-N-(2-isopropyl-4-oxo-7-trifluoromethyl-4H-quinazolin-3-yl)-propionamide;
2-(3-Fluoro-phenyl)-N-(2-isopropyl-4-oxo-4H-quinazolin-3-yl)-propionamide;
2-(4-Chloro-phenyl)-N-(2-isopropyl-4-oxo-4H-quinazolin-3-yl)-propionamide;
N-(2-Isopropyl-4-oxo-7-trifluoromethyl-4H-quinazolin-3-yl)-2-(4-trifluoromethyl-phenyl)-propionamide;
N-(2-Isopropyl-4-oxo-4H-quinazolin-3-yl)-2-(4-methoxy-phenyl)-propionamide;
N-(2-Isopropyl-4-oxo-7-trifluoromethyl-4H-quinazolin-3-yl)-2-(4-methoxy-phenyl)-propionamide;
2-(3,4-Dichloro-phenyl)-N-(2-isopropyl-4-oxo-4H-quinazolin-3-yl)-propionamide;
2-(3-Fluoro-4-methyl-phenyl)-N-(2-isopropyl-4-oxo-7-trifluoromethyl-4H-quinazolin-3-yl)-propionamide;
2-(3-Fluoro-4-methyl-phenyl)-N-(2-isopropyl-4-oxo-4H-quinazolin-3-yl)-propionamide;
2-(4-Isobutyl-phenyl)-N-(2-isopropyl-4-oxo-4H-quinazolin-3-yl)-propionamide;
N-(7-Chloro-2-isopropyl-4-oxo-4H-quinazolin-3-yl)-2-(3-fluoro-phenyl)-propionamide;
N-(6-Chloro-2-isopropyl-4-oxo-4H-quinazolin-3-yl)-2-(3-fluoro-phenyl)-propionamide;
2-(4-Fluoro-phenyl)-N-(2-isopropyl-4-oxo-4H-quinazolin-3-yl)-propionamide;
N-(5-Chloro-2-isopropyl-4-oxo-4H-quinazolin-3-yl)-2-(3-fluoro-phenyl)-propionamide;
N-(8-Chloro-2-isopropyl-4-oxo-4H-quinazolin-3-yl)-2-(3-fluoro-phenyl)-propionamide;
N-(8-Cyano-2-isopropyl-4-oxo-4H-quinazolin-3-yl)-2-(3-fluoro-phenyl)-propionamide;
7-Methyl-bicyclo[4.2.0]octa-1(6),2,4-triene-7-carboxylic acid (2-isopropyl-4-oxo-4H-quinazolin-3-yl)-amide;
N-(2-Isopropylsulfanyl-4-oxo-4H-quinazolin-3-yl)-2-phenyl-propionamide;
2-(4-Chloro-phenyl)-N-(2-isopropylsulfanyl-4-oxo-4H-quinazolin-3-yl)-propionamide;
2-(4-Chloro-phenyl)-N-(2-ethoxy-4-oxo-4H-quinazolin-3-yl)-propionamide;
2-(3,5-Difluoro-phenyl)-N-(2-methylsulfanyl-4-oxo-7-trifluoromethyl-4H-quinazolin-3-yl)-propionamide;
2-Fluoro-N-(2-isopropyl-4-oxo-4H-quinazolin-3-yl)-2-phenyl-propionamide;
(S)-2-Fluoro-N-(2-isopropyl-4-oxo-4H-quinazolin-3-yl)-2-phenyl-propionamide;
(R)-2-Fluoro-N-(2-isopropyl-4-oxo-4H-quinazolin-3-yl)-2-phenyl-propionamide;
(S)-2-Fluoro-N-(2-isopropyl-4-oxo-7-trifluoromethyl-4H-quinazolin-3-yl)-2-phenyl-propionamide; or
(R)-2-Fluoro-N-(2-isopropyl-4-oxo-7-trifluoromethyl-4H-quinazolin-3-yl)-2-phenyl-propionamide;

or a pharmaceutically-acceptable addition salt thereof.

Any combination of two or more of the embodiments described herein is considered within the scope of the present invention.

### DEFINITION OF SUBSTITUENTS

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention a cycloalkyl group designates a cyclic alkyl group, preferably containing of from three to seven carbon atoms (C₃₋₇-cycloalkyl), including cyclopropyl, cyclobutyl, cyclopentyl, cyclohexyl and cycloheptyl.

In the context of this invention halo represents fluoro, chloro, bromo or iodo. Thus a trihalomethyl group represents e.g. a trifluoromethyl group, a trichloromethyl group, and similar trihalo-substituted methyl groups.

In the context of this invention a haloalkyl group designates an alkyl group as defined herein, which alkyl group is substituted one or more times with halo. Preferred haloalkyl groups of the invention include trihalomethyl, preferably trifluoromethyl.

In the context of this invention a hydroxy-alkyl group designates an alkyl group as defined above, which hydroxy-alkyl group is substituted with one or more hydroxy groups. Examples of preferred hydroxy-alkyl groups of the invention include 2-hydroxy-ethyl, 3-hydroxy-propyl, 4-hydroxy-butyl, 5-hydroxy-pentyl and 6-hydroxy-hexyl.

In the context of this invention an alkoxy group designates an “alkyl-O—” group, wherein alkyl is as defined above. Examples of preferred alkoxy groups of the invention include methoxy and ethoxy.

In the context of this invention an alkylthio group designates an “alkyl-S-” group, wherein alkyl is as defined above. Examples of preferred alkoxy groups of the invention include methylthio/methylsylfanyl and ethylthio/ethylsulfanyl.

In the context of this invention an alkyl-carbonyl-amino group designates an “alkyl-CO—NH—” group, wherein alkyl is as defined above. Preferred alkyl-carbonyl-amino groups of the invention include acetamido.

### Pharmaceutically Acceptable Salts

The quinazoline derivatives of the invention may be provided in any form suitable for the intended administration. Suitable forms include pharmaceutically (i.e. physiologically) acceptable salts, and pre- or prodrug forms of the quinazoline derivatives of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzenesulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Examples of pharmaceutically acceptable cationic salts of a chemical compound of the invention include, without limitation, the sodium, the potassium, the calcium, the magnesium, the zinc, the aluminium, the lithium, the choline, the lysine, and the ammonium salt, and the like, of a chemical compound of the invention containing an anionic group. Such cationic salts may be formed by procedures well known and described in the art.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzene-sulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a chemical compound of the invention include, without limitation, the sodium, the potassium, the calcium, the magnesium, the zinc, the aluminium, the lithium, the choline, the lysine, and the ammonium salt, and the like, of a chemical compound of the invention containing an anionic group. Such cationic salts may be formed by procedures well known and described in the art.

### Steric Isomers

The quinazoline derivatives of the present invention may exist in (+) and (−) forms as well as in racemic forms (±). The racemates of these isomers and the individual isomers themselves are within the scope of the present invention.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. Racemic compounds of the present invention can thus be resolved into their optical antipodes, e.g., by fractional crystallisation of d- or I- (tartrates, mandelates, or camphorsulphonate) salts for example.

The quinazoline derivatives of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

### Methods of Preparation

The quinazoline derivatives of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The quinazoline derivatives of the invention have been found useful as modulators of the Kv7 (KCNQ) potassium channels. At present five such channels are known, i.e. the Kᵥ7.1 (KCNQ1) channel, the Kᵥ7.2 (KCNQ2) channel, the Kᵥ7.3 (KCNQ3) channel, the Kᵥ7.4 (KCNQ4) channel, and the Kᵥ7.5 (KCNQ5) channel, and heteromeric combinations hereof. Moreover, the modulatory activity may be inhibitory (i.e. inhibitory activity) or stimulatory (i.e. activating activity).

The modulatory activity may be determined using conventional methods, e.g. binding or activity studies, known in the art, or as described in the working examples.

In a preferred embodiment the quinazoline derivatives of the invention show stimulating activity at Kᵥ7.2, Kᵥ7.3, Kᵥ7.4 and/or Kᵥ7.5 potassium channels, and heteromeric combinations hereof. Preferred compounds of the invention are selective, preferably showing Kᵥ7.4 and/or Kᵥ7.5 potassium channel activation.

Accordingly, the compounds of the invention are considered useful for treatment, prevention or alleviation of a disease or a disorder or a condition of a living animal body, including a human, which disorder, disease or condition is responsive to modulation of a Kv7 potassium channel.

Due to the distribution of Kv7 channels within the organism, Kv7 channel modulators are considered useful for the treatment or alleviation of conditions as diverse as pain, migraine, tension type headache, PNS disorders, CNS disorders, CNS damage caused by trauma, stroke or neurodegenerative illness or diseases, learning and cognitive disorders, motion and motor disorders, multiple sclerosis, heart failure, cardiomyopathia, cardiac disorders, inflammatory diseases, ophthalmic conditions, progressive hearing loss or tinnitus, obstructive or inflammatory airway diseases, for inducing or maintaining bladder control including the treatment or prevention of urinary incontinence.

In a preferred embodiment the compounds of the invention are considered useful for treatment, prevention or alleviation of a disease, disorder or adverse condition of the CNS. In a more specific embodiment, the disease, disorder or condition is an affective disorder, a neuro-physiological disorder, anxiety, depression, a bipolar disorder, mania, a sleep disorder, addiction, an eating disorder, a phobia, Parkinson's disease, a mood disorder, a psychotic disorder, a compulsive behaviour, mania, psychosis or schizophrenia.

In a more preferred embodiment the disease, disorder or condition contemplated according to the invention is anxiety.

In another preferred embodiment the compounds of the invention are considered useful for treatment, prevention or alleviation of a CNS damage caused by trauma or by a spinal cord damage, stroke, a neurodegenerative illness or disease, dementia, Alzheimer's disease, a motor neuron disease, a Parkinson-like motor disorder, multiple sclerosis, amyelotrophic lateral sclerosis (ALS), HIV dementia, Huntington's disease, Pick's disease, torsades de pointes, tremor, muscle spasms, myasthenia gravis, convulsions, ataxia, myokymia, seizures, epilepsy or spasticity.

In a third preferred embodiment the compounds of the invention are considered useful for treatment, prevention or alleviation of pain, including acute and chronic pain, neuropathic pain, central pain, or pain related to diabetic neuropathy, to postherpetic neuralgia, to peripheral nerve injury or drug addiction, migraine and migraine-related disorders and to tension-type headache. In a more specific embodiment the pain is somatic pain, incl. visceral pain or cutaneous pain, or pain caused by inflammation or by infection. In another specific embodiment the pain is neuropathic, e.g. caused by injury to the central or peripheral nervous system, e.g. due to tissue trauma, infection, diabetes, an autoimmune disease, arthritis or neuralgia.

In a fourth preferred embodiment the compounds of the invention are considered useful for treatment, prevention or alleviation of a learning and cognitive disorder, memory dysfunction, memory impairment or age-associated memory loss.

In a fifth preferred embodiment the compounds of the invention are considered useful for treatment, prevention or alleviation of a disease, disorder or condition associated with the heart or skeletal muscle, heart failure, cardiomyopathia, cardiac arrhythmia, cardiac ischaemia or long QT syndrome.

In a sixth preferred embodiment the compounds of the invention are considered useful for treatment, prevention or alleviation of an inflammatory disease or condition, inflammatory bowel disease, Crohn's disease, ulcerative colitis or Creutzfeld-Jacobs disease.

In a seventh preferred embodiment the compounds of the invention are considered useful for treatment, prevention or alleviation of asthma, an obstructive or inflammatory airway disease, an airway hyper reactivity, a pneumoconiosis such as aluminosis, anthracosis, asbestosis, chalicosis, ptilosis, siderosis, silicosis, tabacosis and byssinosis, a chronic obstructive pulmonary disease (COPD), excerbation of airways hyper reactivity or cystic fibrosis.

In an eight preferred embodiment the compounds of the invention are considered useful for treatment, prevention or alleviation of progressive hearing loss or tinnitus, an ophthalmic disorder, a drug-dependence or drug-addiction disorder, hyperactive gastric motility or urinary incontinence.

In a more preferred embodiment the compounds of the invention are considered useful for treatment, prevention or alleviation of pain, neurodegenerative disorders, migraine, bipolar disorders, mania, epilepsy, convulsions, seizures and seizure disorders, anxiety, depression, functional bowel disorders and multiple sclerosis.

In an even more preferred embodiment the compounds of the invention are considered useful for treatment, prevention or alleviation of pain, including mild, moderate or even severe pain of acute, chronic or recurrent character, as well as neuropathic pain and pain caused by migraine, postoperative pain, phantom limb pain, neuropathic pain, chronic headache, tension type headache, central pain, pain related to diabetic neuropathy, to post therapeutic neuralgia, or to peripheral nerve injury.

In a most preferred embodiment the compounds of the invention are considered useful for treatment, prevention or alleviation of pain, neuropathic pain, epilepsy or anxiety.

### Pharmaceutical Compositions

Viewed from one aspect the invention relates to the use of a quinazoline derivative of the invention, or a pharmaceutically-acceptable addition salt thereof, for the manufacture of a pharmaceutical composition for the treatment, prevention or alleviation of a disease or a disorder or a condition of a mammal, including a human, which disease, disorder or condition is responsive to modulation of Kv7 channels.

Viewed from another aspect, the invention provides pharmaceutical compositions comprising a therapeutically-effective amount of a quinazoline derivative of the invention, or a pharmaceutically-acceptable addition salt thereof, together with at least one pharmaceutically-acceptable carrier or diluent, for the treatment, prevention or alleviation of a disease or a disorder or a condition that is responsive to modulation of Kv7 channels.

While a quinazoline derivative for use according to the invention may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising a quinazoline derivative of the invention, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route which suite the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition may be prepared by the skilled person using standard and conventional techniques appropriate for the desired formulation. When desired, compositions adapted to give sustained release of the active ingredient may be employed.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

In a preferred embodiment the pharmaceutical composition of the invention comprises a therapeutically effective amount of N-(2-Isopropyl-4-oxo-4H-quinazolin-3-yl)-2-phenyl-propionamide; or a pharmaceutically-acceptable addition salt thereof, together with one or more adjuvants, excipients, carriers and/or diluents.

### Methods of Therapy

In another aspect the invention provides a method for the treatment, prevention or alleviation of a disease or a disorder or a condition of a living animal body, including a human, which disease, disorder or condition is responsive to activation of Kᵥ7 channels, and which method comprises administering to such a living animal body, including a human, in need thereof an effective amount of a quinazoline derivative of the invention.

The preferred medical indications contemplated according to the invention are those stated above.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Preferred ranges are from about 0.001 to about 1 mg/kg i.v. and from about 0.1 to about 10 mg/kg p.o.

### EXAMPLES

The invention is further illustrated with reference to the following examples, which are not intended to be in any way limiting to the scope of the invention as claimed.

### Example 1

### Preparative Example

The compounds of the invention may be synthesised as outlined in general terms and described in more details below.

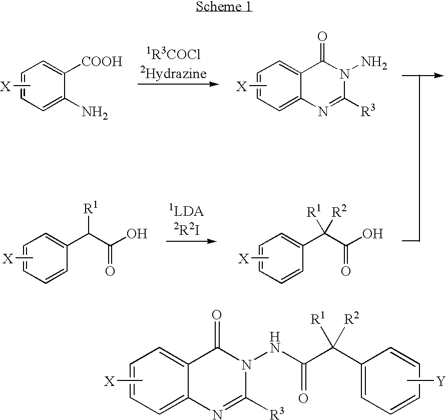

### 3-Amino-2-ethyl-7-fluoro-3H-quinazolin-4-one (Intermediate compound)

A solution of 2-amino-4-fluorobenzoic acid (1 g, 6.4 mmol) in THF (40 mL) was added pyridine (3.2 mL, 38.7 mmol) and propionylchloride (2.2 mL, 25.8 mmol) and then heated to reflux over night after and placed on an ice-water bath. The reaction mixture was added hydrazine hydrate (3.8 mL, 77.4 mmol) and stirred at 0° C. for 15 minutes, then at room temperature for 1 hour and eventually at reflux for 1 hour after which it was left to cool to room temperature over night. The mixture was evaporated onto silica gel and purified on CombiFlash™ sq16 (40 g silica gel column, eluent: 100% benzine (Bp.=80-100° C.) to 100% EtOAc) to give 0.7 g (52%) of pure title compound.

### Method A

### N-(2-Ethyl-7-fluoro-4-oxo-4H-quinazolin-3-yl)-2-phenyl-propionamide (Compound A1)

A clear solution of DL-2-phenylpropionic acid (70 uL, 0.511 mmol.), 1-(3-dimethylaminopropyl)-3-ethylcarbodiimide, hydrochloride (EDC HCl, 128 mg; 0.668 mmol) and 4-dimethylaminopyridine (DMAP, 59 mg; 0.483 mmol) in CH₂Cl₂ (3 mL) was stirred for 5 minutes in a dry flask under N₂, after which 3-amino-2-ethyl-7-fluoro-3H-quinazolin-4-one (100 mg, 0.483 mmol) was added and the mixture was stirred over night at room temperature. The reaction mixture was added EDC HCl (60 mg; 0.3 mmol) and after 10 minutes 35 μL acid (0.26 mmol.) after which it was left with stirring over night. This procedure was repeated two more times after which the reaction mixture was filtered through Hydromatrix [2 g, treated with 4 mL 2M NaOH (aq.)+2 g, treated with 4 mL 2M HCl (aq.)] and a Na₂SO₄ filter. The crude product was further purified by preparative LC-MS to give 29 mg (18%) of pure title compound. Mp. 57-63° C.

### Method B

### N-(2-Isopropyl-4-oxo-4H-quinazolin-3-yl)-2-phenyl-propionamide (Compound B1)

To a solution of DL-2-phenylpropionic acid (0.8 mL; 5.9 mmol) in dry THF (50 mL) was added three drops of dry dimethylformamide and—carefully—oxalyl chloride (0.6 mL; 6.9 mmol) and the mixture was left with stirring for 30 minutes after which it was heated to 50° C. for 1 hour. Pyridine (0.4 mL; 4.9 mmol) was added giving immediate precipitation, then 3-amino-2-isopropyl-3H-quinazolin-4-one (1.00 g; 4.92 mmol) and more pyridine (0.8 mL; 9.8 mmol) and the mixture was stirred over night after which sat. NaHCO₃ (30 mL) was added and the reaction mixture was left with stirring for 30 minutes, added 100 mL EtOAc and 20 mL H₂O. The organic phase was isolated, washed with 20 mL H₂O added a little brine to help phase separation, dried (MgSO₄), filtered and evaporated onto Celite™. The crude product was purified by CombiFlash™ SQ16 [eluent: benzine (bp.=80-100° C.)/EtOAc=9:1 going to 100% EtOAc over 18 minutes] to give after evaporation a sticky foam. This was re-dissolved in diethyl ether and evaporated at room temperature in vacuo to give 1.4 g (85%) of a white crystalline compound. Mp. 48-55° C.

Using Method B the following compounds were synthesized:

### N-(2-Isopropyl-4-oxo-4H-quinazolin-3-yl)-3-methyl-2-phenyl-butyramide (Compound B2)

Mp. 188-193° C.; LC-ESI-HRMS of [M+H]+ shows 364.2006 Da. Calc. 364.202502 Da, dev. −5.2 ppm.

### (S)-N-(2-Isopropyl-4-oxo-4H-quinazolin-3-yl)-2-phenyl-propionamide (Compound B3)

Specific rotation: [α]₅₈₉=200° (MeOH; n=2); LC-ESI-HRMS of [M+H]+shows 336.1724 Da. Calc. 336.171202 Da.

### N-(2-Isopropyl-4-oxo-4H-quinazolin-3-yl)-2,3-diphenyl-propionamide (Compound B4)

LC-ESI-HRMS of [M+H]+ shows 412.201 Da. Calc. 412.202502 Da.

### Bicyclo[4.2.0]octa-1,3,5-triene-7-carboxylic acid (2-isopropyl-4-oxo-4H-quinazolin-3-yl)-amide (Compound B5)

LC-ESI-HRMS of [M+H]+ shows 334.1543 Da. Calc. 334.155552 Da.

### N-(2-Isopropyl-4-oxo-4H-quinazolin-3-yl)-2-p-tolyl-propionamide (Compound B6)

LC-ESI-HRMS of [M+H]+ shows 350.1862 Da. Calc. 350.186852 Da.

### 2-Cyclohexyl-N-(2-isopropyl-4-oxo-4H-quinazolin-3-yl)-2-phenyl-acetamide (Compound B7)

LC-ESI-HRMS of [M+H]+ shows 404.235 Da. Calc. 404.233802 Da;

### 2-(3-Benzoyl-phenyl)-N-(2-isopropyl-4-oxo-4H-quinazolin-3-yl)-propionamide (Compound B8)

LC-ESI-HRMS of [M+H]+ shows 440.1974 Da. Calc. 440.197417 Da.

### 1-Phenyl-cyclopropanecarboxylic acid (2-isopropyl-4-oxo-4H-quinazolin-3-yl)-amide (Compound B9)

LC-ESI-HRMS of [M+H]+ shows 348.1693 Da. Calc. 348.171202 Da.

### 2-(3,4-Dimethoxy-phenyl)-N-(2-isopropyl-4-oxo-4H-quinazolin-3-yl)-propionamide (Compound B10)

LC-ESI-HRMS of [M+H]+ shows 396.1914 Da. Calc. 396.192332 Da.

### (R)-N-(2-Isopropyl-4-oxo-4H-quinazolin-3-yl)-2-phenyl-propionamide (Compound B11)

LC-ESI-HRMS of [M+H]+ shows 336.1707 Da. Calc. 336.171202 Da.

### N-(2-Isopropyl-4-oxo-4H-quinazolin-3-yl)-2-phenyl-isobutyramide (Compound B12)

LC-ESI-HRMS of [M+H]+ shows 350.186 Da. Calc. 350.186852 Da.

### 2-(4-Chloro-phenyl)-N-(2-isopropyl-4-oxo-7-trifluoromethyl-4H-quinazolin-3-yl)-propionamide (Compound B13)

LC-ESI-HRMS of [M+H]+ shows 438.1205 Da. Calc. 438.119614 Da.

### N-(2-isopropyl-4-oxo-4H-quinazolin-3-yl)-2-(4-trifluoromethyl-phenyl)-propionamide (Compound B14)

LC-ESI-HRMS of [M+H]+ shows 404.1569 Da. Calc. 404.158586 Da.

### 2-(3,4-Dichloro-phenyl)-N-(2-isopropyl-4-oxo-7-trifluoromethyl-4H-quinazolin-propionamide (Compound B15)

LC-ESI-HRMS of [M+H]+ shows 472.0783 Da. Calc. 472.080642 Da.

### 2-(3-Fluoro-phenyl)-N-(2-isopropyl-4-oxo-4H-quinazolin-3-yl)-propionamide (Compound B16)

LC-ESI-HRMS of [M+H]+ shows 354.1628 Da. Calc. 354.16178 Da.

### 2-(4-Chloro-phenyl)-N-(2-isopropyl-4-oxo-4H-quinazolin-3-yl)-propionamide (Compound B17)

LC-ESI-HRMS of [M+H]+ shows 370.1302 Da. Calc. 370.13223 Da;

### N-(2-Isopropyl-4-oxo-7-trifluoromethyl-4H-quinazolin-3-yl)-2-(4-trifluoromethyl-phenyl)-propionamide (Compound B18)

LC-ESI-HRMS of [M+H]+ shows 472.1476 Da. Calc. 472.14597 Da.

### N-(2-Isopropyl-4-oxo-4H-quinazolin-3-yl)-2-(4-methoxy-phenyl)-propionamide (Compound B19)

### N-(2-Isopropyl-4-oxo-7-trifluoromethyl-4H-quinazolin-3-yl)-2-(4-methoxy-phenyl)-propionamide (Compound B20)

### 2-(3,4-Dichloro-phenyl)-N-(2-isopropyl-4-oxo-4H-quinazolin-3-yl)-propionamide (Compound B21)

### 2-(3-Fluoro-4-methyl-phenyl)-N-(2-isopropyl-4-oxo-7-trifluoromethyl-4H-quinazolin-3-yl)-propionamide (Compound B22)

### 2-(3-Fluoro-4-methyl-phenyl)-N-(2-isopropyl-4-oxo-4H-quinazolin-3-yl)-propionamide (Compound B23)

LC-ESI-HRMS of [M+H]+ shows 368.1772 Da. Calc. 368.17743 Da.

### 2-(4-Isobutyl-phenyl)-N-(2-isopropyl-4-oxo-4H-quinazolin-3-yl)-propionamide (Compound B24)

LC-ESI-HRMS of [M+H]+ shows 392.2328 Da.

### N-(7-Chloro-2-isopropyl-4-oxo-4H-quinazolin-3-yl)-2-(3-fluoro-phenyl)-propionamide (Compound B25)

LC-ESI-HRMS of [M+H]+ shows 388.1244 Da.

### N-(6-Chloro-2-isopropyl-4-oxo-4H-quinazolin-3-yl)-2-(3-fluoro-phenyl)-propionamide (Compound B26)

LC-ESI-HRMS of [M+H]+ shows 388.121 Da.

### 2-(4-Fluoro-phenyl)-N-(2-isopropyl-4-oxo-4H-quinazolin-3-yl)-propionamide (Compound B27)

LC-ESI-HRMS of [M+H]+ shows 354.1604 Da.

### N-(5-Chloro-2-isopropyl-4-oxo-4H-quinazolin-3-yl)-2-(3-fluoro-phenyl)-propionamide (Compound B28)

LC-ESI-HRMS of [M+H]+ shows 388.122 Da;

### N-(8-Chloro-2-isopropyl-4-oxo-4H-quinazolin-3-yl)-2-(3-fluoro-phenyl)-propionamide (Compound B29)

LC-ESI-HRMS of [M+H]+ shows 388.1222 Da.

### N-(8-Cyano-2-isopropyl-4-oxo-4H-quinazolin-3-yl)-2-(3-fluoro-phenyl)-propionamide (Compound B30)

### 7-Methyl-bicyclo[4.2.0]octa-1(6),2,4-triene-7-carboxylic acid (2-isopropyl-4-oxo-4H-quinazolin-3-yl)-amide (Compound B31)

LC-ESI-HRMS of [M+H]+ shows 348.1711 Da. 7-Methyl-bicyclo[4.2.0]octa-1(6),2,4-triene-7-carboxylic acid was synthesized according to J. Chem. Soc. Perkin Trans. I 1985 p. 2153.

### Example 2

### Preparative Example

Sulfur analogues may be synthesised the following way:

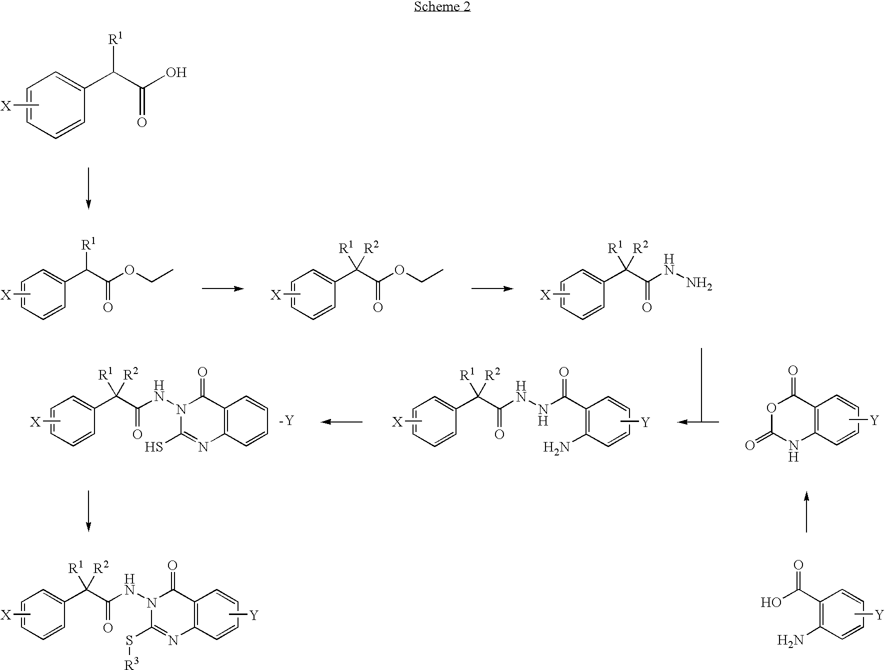

### (4-Chloro-phenyl)-acetic acid ethyl ester (Intermediate compound)

A solution of 4-chlorophenylacetic acid (28.51 g, 167 mmol) in ethanol

(99%, 85 mL) was added conc. sulfuric acid (15 mL) and refluxed for 4 hours after which the reaction mixture was concentrated to 40 mL and poured into water. The aqueous phase was extracted with diethyl ether, dried using MgSO₄, filtered and evaporated in vacuo. The crude product was further purified by distillation (approx. 100° C./1 Torr) to give 27.2 g (82%) of the title compound.

### 2-(4-Chloro-phenyl)-butyric acid ethyl ester (Intermediate compound)

To a solution of diisopropylamine (3.9 mL, 28.1 mmol) in dry THF (50 mL) under N₂ at −78° C. was slowly added butyl lithium (2.5 M in THF, 11.5 mL, 28.1 mmol) such that the temperature did not exceed −65° C. The clear solution was stirred for 30 min at −78° C., and then added (4-chloro-phenyl)-acetic acid ethyl ester (5 g, 25.3 mmol in 5 mL THF). The reaction mixture was stirred at −70° C. for 45 minutes after which the dry-ice/acetone bath was replaced by an ice-water bath in order to warm to 0° C. After stirring for 30 minutes, the mixture was re-cooled to −78° C. and iodoethane (3.4 mL, 42.2 mmol) was added. The reaction mixture was allowed to warm to room temperature over night after which a NH₄Cl solution (half saturated aqueous solution) was added. The aqueous slurry was extracted twice with EtOAc, the combined organic phases was, dried using MgSO₄, filtered and evaporated to dryness leaving 7.7 g of crude title compound which was taken as such for next step.

### 2-(4-Chloro-phenyl)-butyric acid hydrazide (Intermediate compound)

A solution of 2-(4-Chloro-phenyl)-butyric acid ethyl ester (max. 25.3 mmol) in ethanol (99%, 30 mL) was added hydrazine hydrate (2.5 meq, 50.6 mmol) and heated at reflux over night. The reaction mixture was heated at reflux for another 24 hours and while added hydrazine hydrate in two portions (2×2.5 mL, 100 mmol). The mixture was then allowed to cool to room temperature, evaporated in vacuo and added water. The formed precipitate was isolated by filtration, washed with water and dried in vacuo to leave 4.4 g (82%) of product as light yellow crystals.

### 1H-Benzo[d][1,3]oxazine-2,4-dione (Intermediate compound)

A solution of 2-amino benzoic acid (10 g, 72.92 mmol) and pyridine 23.6 mmol, 292 mmol) in a mixture of CH₂Cl₂ (100 mL) and CH₃CN (100 mL) under N₂ atmosphere was carefully added a solution of diphosgene (4.4 mL, 36.5 mmol) in CH₂Cl₂ (25 mL). The reaction mixture was then heated to 50° C. over night after which it was cooled and poured into ethyl acetate and a half saturated aqueous NaCl solution. The organic phase was dried using MgSO₄, filtered and evaporated to dryness. The crude product was triturated in diethyl ether to give 7.5 g (63%) of the title compound.

### 2-Amino-benzoic acid N′-[2-(4-chloro-phenyl)-butyryl]-hydrazide (Intermediate Compound)

A solution of 1H-benzo[d][1,3]oxazine-2,4-dione (0.87 g, 5.1 mmol) in glacial acetic acid (20 mL) was added 2-(4-chloro-phenyl)-butyric acid hydrazide (1.0 g, 4.7 mmol) and the mixture was stirred at room temperature for 3 hours after which the reaction mixture was poured into water. The aqueous solution was made alkaline using saturated aqueous sodium carbonate solution after which it was extracted with ethyl acetate, dried using MgSO₄, filtered and evaporated to give 1.65 g of relatively title compound which was used as such for the next step.

### 2-(4-Chloro-phenyl)-N-(2-mercapto-4-oxo-4H-quinazolin-3-yl)-butyramide (Intermediate Compound)

A solution of 2-Amino-benzoic acid N′-[2-(4-chloro-phenyl)-butyryl]-hydrazide (max. 4.7 mmol) in ethanol (99%, 25 mL) was added ethylxanthic acid potassium salt (1.1 g, 7.1 mmol) and heated to reflux over night. The reaction mixture was heated at reflux for another 24 hours and while added ethylxanthic acid potassium salt in two portions (2×1.1 g, 14.2 mmol). The reaction mixture was evaporated in vacuo and the remanence added water and ethyl acetate. The pH of the aqueous phase was adjusted to pH=7 using 1 M HCl (aq.) and the organic phase isolated. The aqueous phase was extracted twice with ethyl acetate and the combined organic phases were dried using MgSO₄, filtered and evaporated to in vacuo to give a yellow foam. The crude product was triturated in diethyl ether and dried in vacuo to give 0.7 g (40%) of the title compound used as such for the next step.

### 2-(4-Chloro-phenyl)-N-(2-isopropylsulfanyl-4-oxo-4H-quinazolin-3-yl)-butyramide (Compound C1)

To a solution of 2-(4-Chloro-phenyl)-N-(2-mercapto-4-oxo-4H-quinazolin-3-yl)-butyramide (0.7 g, 1.87 mmol) in ethanol (99%, 20 mL) was added CsOH (aq., 3M, 0.69 mL, 2.1 mmol) and 2-bromopropane (0.19 mL, 2.1 mmol). The reaction mixture was stirred at 60° C. for 4 hours, added more 2-bromopropane (0.8 mL, 8.5 mmol) and left with stirring over night at 60° C. The mixture was allowed to cool to room temperature, added water and acidified using 1 M HCl (aq.). The aqueous mixture was extracted twice with ethyl acetate and the combined organic phases was dried (MgSO₄), filtered, and evaporated in vacuo to give 0.7 g pure product which was further purified by column chromatography on a Combiflash sq16 system (40 g silica gel column, eluent 100% benzine (Bp. 80-100° C.) to 100% ethyl acetate to give 0.38 g (49%) pure product. Mp. 158-163° C.

In a similar manner the following compounds were synthesised:

### 2-(3,5-Difluoro-phenyl)-N-(2-isopropylsulfanyl-4-oxo-7-trifluoromethyl-4H-quinazolin-3-yl)-propionamide (Compound C2)

LC-ESI-HRMS of [M+H]+ shows 472.1132 Da. Calc. 472.1118, dev. 2.9 ppm.

### N-(2-Isopropylsulfanyl-4-oxo-4H-quinazolin-3-yl)-2-phenyl-propionamide (Compound C3)

LC-ESI-HRMS of [M+H]+ shows 368.1451 Da.

### 2-(4-Chloro-phenyl)-N-(2-isopropylsulfanyl-4-oxo-4H-quinazolin-3-yl)-propionamide (Compound C4)

LC-ESI-HRMS of [M+H]+ shows 402.1054 Da.

### 2-(4-Chloro-phenyl)-N-(2-ethoxy-4-oxo-4H-quinazolin-3-yl)-propionamide (Compound C5)

LC-ESI-HRMS of [M+H]+ shows 372.111 Da. Calc. 372.111495 Da. From the above reaction was also isolated 12.5% of the title compound; and

### 2-(3,5-Difluoro-phenyl)-N-(2-methylsulfanyl-4-oxo-7-trifluoromethyl-4H-quinazolin-3-yl)-propionamide (Compound C6)

### Example 3

### Preparative Example

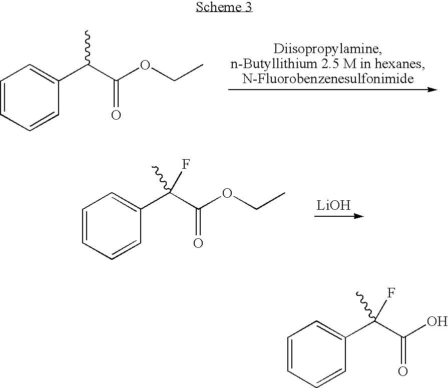

### 2-Fluoro-2-phenyl-propionic acid ethyl ester (Intermediate compound)

A solution of diisopropylamine (18.5 mL; 131.3 mmol) in THF (150 mL) was cooled to −78° C. and added n-butyllithium (2.5 M in hexanes; 52.5 mL; 131.3 mmol) after which 2-phenyl-propionic acid ethyl ester (18 g, 101 mmol) was added over 15 min. The mixture was stirred at −78° for 30 min, then at 0° C. for 30 min and then recooled to −78° again before N-fluorobenzenesulfonimide (35 g; 111 mmol in 120 mL dry THF) was added. After complete addition the reaction mixture was allowed to reach room temperature. The reaction mixture was added glacial acetic acid (8 mL), brine and EtOAc. The organic phase was isolated and the aqueous phase was washed with EtOAc. The combined organics were washed with 5% Na₂CO₃, brine, dried on Na₂SO₄ and evaporated to give 20.1 g light brown oil. The crude product was diluted with petrol ether+EtOAc (2:1) and filtered over a plug of silica to give after evaporation to dryness: 19 g of title product (96%).

### 2-Fluoro-2-phenyl-propionic acid (Intermediate compound)

To a solution of 2-fluoro-2-phenyl-propionic acid ethyl ester (19 g; 96.8 mmol) in H₂O/THF (100 mL+100 mL) was added lithium hydroxide monohydrate (20.3 g; 484 mmol) and stirred for 2 h at room temperature. The THF was removed by evaporation and pH adjusted to pH=1 using 6 M HCl (aq). The water mixture is extracted with EtOAc and the organics were washed with brine, dried on Na₂SO₄ and evaporated to dryness to give 16.4 g (quantitative) title compound as an orange oil.

### 2-Fluoro-N-(2-isopropyl-4-oxo-4H-quinazolin-3-yl)-2-phenyl-propionamide (Compound D1)

LC-ESI-HRMS of [M+H]+ shows 354.1599 Da. Calc. 354.16178 Da. The compound was synthesized as described in Example 1.

### (S)-2-Fluoro-2-phenyl-propionic acid and (R)-2-Fluoro-2-phenyl-propionic acid (Intermediate Compounds)

The isomers were resolved by the method described in J. Fluor. Chem. 1993 60 225-232, with the modification, that (R)-(+)-1-(1-Naphtyl)ethylamine and (S)-(+)-1-(1-Naphtyl)ethylamine was used for the resolvation step which was performed 3 times for each isomer to give products with 95% ee and 94% ee respectively. Specific rotation: +25.25 (0.1 g in 10 ml EtOH) and −22.15 (0.1 g in 10 ml EtOH) respectively.

### (S)-2-Fluoro-N-(2-isopropyl-4-oxo-4H-quinazolin-3-yl)-2-phenyl-propionamide (Compound D2)

The title compound was synthesized according to Example 1. LC-ESI-HRMS of [M+H]+ shows 354.1613 Da. Calc. 354.16178 Da.

### (R)-2-Fluoro-N-(2-isopropyl-4-oxo-4H-quinazolin-3-yl)-2-phenyl-propionamide (Compound D3)

LC-ESI-HRMS of [M+H]+ shows 354.1637 Da. Calc. 354.16178 Da. Synthesized according to Example 1.

### (S)-2-Fluoro-N-(2-isopropyl-4-oxo-7-trifluoromethyl-4H-quinazolin-3-yl)-2-phenyl-propionamide (Compound D4)

LC-ESI-HRMS of [M+H]+ shows 422.1476 Da. Calc. 422.149164 Da. Synthesized according to Example 1.

### (R)-2-Fluoro-N-(2-isopropyl-4-oxo-7-trifluoromethyl-4H-quinazolin-3-yl)-2-phenyl-propionamide (Compound D5)

LC-ESI-HRMS of [M+H]+ shows 422.1472 Da. Calc. 422.149164 Da. Synthesized according to Example 1.

### Example 4

### Biological Activity

In a standard patch-clamp set-up, e.g. as outlined in International Patent Publication WO 2004/080377, using HEK293 cell lines stably expressing the human Kᵥ7₂₊₃ channels, the compounds of the invention were found to be activators of the channels at various concentrations at various degrees. For example, at a concentration of at 3 μM, Compound B16 shows 60% activation and Compound B20 shows 87% activation, when compared to control.

1-18. (canceled)

19. A quinazoline derivative of Formula I 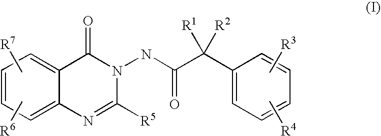
any of its stereoenantiomers or any mixture of its stereoenantiomers, or a pharmaceutically-acceptable addition salt thereof, or an N-oxide thereof, wherein
R¹ represents hydrogen or alkyl; and
R² represents alkyl, cycloalkyl, halo, haloalkyl, hydroxyalkyl, hydroxy, alkoxy, phenyl, phenylalkyl, amino, alkyl-carbonyl-amino, cyano or nitro; or
R¹ and R² together with the carbon atom to which they are attached form a cycloalkyl group; or
R¹ represents hydrogen; and
R² together with R³ attached in ortho-position on the aromatic ring form a —(CH₂)ₙ— bridge, wherein n is 1, 2 or 3;
R³ and R⁴, independently of each other, represent hydrogen, alkyl, cycloalkyl, halo, haloalkyl, hydroxy, alkoxy, haloalkoxy, amino, alkyl-carbonyl-amino, alkyl-sulfonyl, phenyl, benzoyl, cyano or nitro; or
R³ and R⁴ together form a methylenedioxy group; or
R³ attached in ortho-position on the aromatic ring and together with R² form a —(CH₂)ₙ— bridge, wherein n is 1, 2 or 3; and
R⁴ is as defined above;
R⁵ represents alkyl, cycloalkyl, alkoxy, alkylthio or phenyl; and
R⁶ and R⁷, independently of each other, represent hydrogen, alkyl, cycloalkyl, halo, haloalkyl, hydroxy, alkoxy, haloalkoxy, amino, alkyl-carbonyl-amino(acetamido), nitro, cyano or phenyl;
provided, however, that if R¹ is hydrogen, R² is methyl, R³ and R⁴ represent hydrogen, R⁵ is isopropyl, and R⁶ and R⁷ represent hydrogen, then the compound it is not a quinazoline derivative racemate but the R- or S-enantiomer of the quinazoline derivative.

20. The quinazoline derivative of claim 19, or an N-oxide thereof, or a pharmaceutically-acceptable addition salt thereof, wherein
R¹ represents hydrogen or alkyl.

21. The quinazoline derivative of claim 19, or an N-oxide thereof, or a pharmaceutically-acceptable addition salt thereof, wherein
R² represents alkyl, cycloalkyl, halo, haloalkyl, hydroxyalkyl, hydroxy, alkoxy, phenyl, phenylalkyl, amino, alkyl-carbonyl-amino, cyano or nitro.

22. The quinazoline derivative of claim 19, or an N-oxide thereof, or a pharmaceutically-acceptable addition salt thereof, wherein
R¹ represents hydrogen or methyl; and
R² represents methyl, ethyl, isopropyl, cyclohexyl, fluoro or benzyl.

23. The quinazoline derivative of claim 19, or an N-oxide thereof, or a pharmaceutically-acceptable addition salt thereof, wherein
R¹ and R² together with the carbon atom to which they are attached form a cycloalkyl group.

24. The quinazoline derivative of claim 19, or an N-oxide thereof, or a pharmaceutically-acceptable addition salt thereof, wherein
R¹ represents hydrogen; and
R² together with R³ attached in ortho-position on the aromatic ring form a —(CH₂)ₙ— bridge, wherein n is 1, 2 or 3.

25. The quinazoline derivative of claim 19, or an N-oxide thereof, or a pharmaceutically-acceptable addition salt thereof, wherein
R³ and R⁴, independently of each other, represent hydrogen, alkyl, cycloalkyl, halo, haloalkyl, hydroxy, alkoxy, haloalkoxy, amino, alkyl-carbonyl-amino, alkyl-sulfonyl, phenyl, benzoyl, cyano or nitro; or
R³ and R⁴ together form a methylenedioxy group.

26. The quinazoline derivative of claim 19, or an N-oxide thereof, or a pharmaceutically-acceptable addition salt thereof, wherein
R³ attached in ortho-position on the aromatic ring and together with R² form a —(CH₂)ₙ— bridge, wherein n is 1, 2 or 3; and
R⁴ is as defined in claim 7.

27. The quinazoline derivative of claim 19, or a pharmaceutically-acceptable addition salt thereof, wherein
R⁵ represents alkyl, cycloalkyl, alkoxy, alkylthio or phenyl.

28. The quinazoline derivative of claim 19, or an N-oxide thereof, or a pharmaceutically-acceptable addition salt thereof, wherein
R⁶ and R⁷, independently of each other, represent hydrogen, alkyl, cycloalkyl, halo, haloalkyl, hydroxy, alkoxy, haloalkoxy, amino, alkyl-carbonyl-amino(acetamido), nitro, cyano or phenyl.

29. The quinazoline derivative of claim 19, which is
N-(2-Isopropyl-4-oxo-4H-quinazolin-3-yl)-3-methyl-2-phenyl-butyramide;
2-(4-Chloro-phenyl)-N-(2-isopropylsulfanyl-4-oxo-4H-quinazolin-3-yl)-butyramide;
2-(3,5-Difluoro-phenyl)-N-(2-isopropylsulfanyl-4-oxo-7-trifluoromethyl-4H-quinazolin-3-yl)-propionamide;
N-(2-Ethyl-7-fluoro-4-oxo-4H-quinazolin-3-yl)-2-phenyl-propionamide;
(S)-N-(2-Isopropyl-4-oxo-4H-quinazolin-3-yl)-2-phenyl-propionamide;
N-(2-Isopropyl-4-oxo-4H-quinazolin-3-yl)-2,3-diphenyl-propionamide;
Bicyclo[4.2.0]octa-1,3,5-triene-7-carboxylic acid (2-isopropyl-4-oxo-4H-quinazolin-3-yl)-amide;
N-(2-Isopropyl-4-oxo-4H-quinazolin-3-yl)-2-p-tolyl-propionamide;
2-Cyclohexyl-N-(2-isopropyl-4-oxo-4H-quinazolin-3-yl)-2-phenyl-acetamide;
2-(3-Benzoyl-phenyl)-N-(2-isopropyl-4-oxo-4H-quinazolin-3-yl)-propionamide;
1-Phenyl-cyclopropanecarboxylic acid (2-isopropyl-4-oxo-4H-quinazolin-3-yl)-amide;
2-(3,4-Dimethoxy-phenyl)-N-(2-isopropyl-4-oxo-4H-quinazolin-3-yl)-propionamide;
(R)-N-(2-Isopropyl-4-oxo-4H-quinazolin-3-yl)-2-phenyl-propionamide;
N-(2-Isopropyl-4-oxo-4H-quinazolin-3-yl)-2-phenyl-isobutyramide;
2-(4-Chloro-phenyl)-N-(2-isopropyl-4-oxo-7-trifluoromethyl-4H-quinazolin-3-yl)-propionamide;
N-(2-Isopropyl-4-oxo-4H-quinazolin-3-yl)-2-(4-trifluoromethyl-phenyl)-propionamide;
2-(3,4-Dichloro-phenyl)-N-(2-isopropyl-4-oxo-7-trifluoromethyl-4H-quinazolin-3-yl)-propionamide;
2-(3-Fluoro-phenyl)-N-(2-isopropyl-4-oxo-4H-quinazolin-3-yl)-propionamide;
2-(4-Chloro-phenyl)-N-(2-isopropyl-4-oxo-4H-quinazolin-3-yl)-propionamide;
N-(2-Isopropyl-4-oxo-7-trifluoromethyl-4H-quinazolin-3-yl)-2-(4-trifluoromethyl-phenyl)-propionamide;
N-(2-Isopropyl-4-oxo-4H-quinazolin-3-yl)-2-(4-methoxy-phenyl)-propionamide;
N-(2-Isopropyl-4-oxo-7-trifluoromethyl-4H-quinazolin-3-yl)-2-(4-methoxy-phenyl)-propionamide;
2-(3,4-Dichloro-phenyl)-N-(2-isopropyl-4-oxo-4H-quinazolin-3-yl)-propionamide;
2-(3-Fluoro-4-methyl-phenyl)-N-(2-isopropyl-4-oxo-7-trifluoromethyl-4H-quinazolin-3-yl)-propionamide;
2-(3-Fluoro-4-methyl-phenyl)-N-(2-isopropyl-4-oxo-4H-quinazolin-3-yl)-propionamide;
2-(4-Isobutyl-phenyl)-N-(2-isopropyl-4-oxo-4H-quinazolin-3-yl)-propionamide;
N-(7-Chloro-2-isopropyl-4-oxo-4H-quinazolin-3-yl)-2-(3-fluoro-phenyl)-propionamide;
N-(6-Chloro-2-isopropyl-4-oxo-4H-quinazolin-3-yl)-2-(3-fluoro-phenyl)-propionamide;
2-(4-Fluoro-phenyl)-N-(2-isopropyl-4-oxo-4H-quinazolin-3-yl)-propionamide;
N-(5-Chloro-2-isopropyl-4-oxo-4H-quinazolin-3-yl)-2-(3-fluoro-phenyl)-propionamide;
N-(8-Chloro-2-isopropyl-4-oxo-4H-quinazolin-3-yl)-2-(3-fluoro-phenyl)-propionamide;
N-(8-Cyano-2-isopropyl-4-oxo-4H-quinazolin-3-yl)-2-(3-fluoro-phenyl)-propionamide;
7-Methyl-bicyclo[4.2.0]octa-1(6),2,4-triene-7-carboxylic acid (2-isopropyl-4-oxo-4H-quinazolin-3-yl)-amide;
N-(2-Isopropylsulfanyl-4-oxo-4H-quinazolin-3-yl)-2-phenyl-propionamide;
2-(4-Chloro-phenyl)-N-(2-isopropylsulfanyl-4-oxo-4H-quinazolin-3-yl)-propionamide;
2-(4-Chloro-phenyl)-N-(2-ethoxy-4-oxo-4H-quinazolin-3-yl)-propionamide;
2-(3,5-Difluoro-phenyl)-N-(2-methylsulfanyl-4-oxo-7-trifluoromethyl-4H-quinazolin-3-yl)-propionamide;
2-Fluoro-N-(2-isopropyl-4-oxo-4H-quinazolin-3-yl)-2-phenyl-propionamide;
(S)-2-Fluoro-N-(2-isopropyl-4-oxo-4H-quinazolin-3-yl)-2-phenyl-propionamide;
(R)-2-Fluoro-N-(2-isopropyl-4-oxo-4H-quinazolin-3-yl)-2-phenyl-propionamide;
(S)-2-Fluoro-N-(2-isopropyl-4-oxo-7-trifluoromethyl-4H-quinazolin-3-yl)-2-phenyl-propionamide; or
(R)-2-Fluoro-N-(2-isopropyl-4-oxo-7-trifluoromethyl-4H-quinazolin-3-yl)-2-phenyl-propionamide;
or an N-oxide thereof, any of its stereoenantiomers or any mixture of its stereoenantiomers,
or a pharmaceutically-acceptable addition salt thereof.

29. A pharmaceutical composition comprising a therapeutically effective amount of the quinazoline derivative of claim 19, or an N-oxide thereof, any of its stereoenantiomers or any mixture of its stereoenantiomers, or a pharmaceutically-acceptable addition salt thereof.

30. A method of treatment, prevention or alleviation of a disease or a disorder or a condition of a living animal body, including a human, which disorder, disease or condition is responsive to activation of Kᵥ7 channels, which method comprises the step of administering to such a living animal body in need thereof, a therapeutically effective amount of the quinazoline derivative of claim 19, or an N-oxide thereof, any of its stereoenantiomers or any mixture of its stereoenantiomers, or a pharmaceutically-acceptable addition salt thereof.

31. The method according to claim 30, wherein the disease, disorder or condition is pain, neurodegenerative disorders, migraine, bipolar disorders, mania, epilepsy, convulsions, seizures and seizure disorders, anxiety, depression, functional bowel disorders and multiple sclerosis.

32. The method according to claim 30, wherein the disease, disorder or condition is pain, mild, moderate or severe pain, acute, chronic or recurrent pain, neuropathic pain, pain caused by migraine, postoperative pain, phantom limb pain, neuropathic pain, chronic headache, tension type headache, central pain, pain related to diabetic neuropathy, to post therapeutic neuralgia, or to peripheral nerve injury.

33. The method according to claim 30, wherein the disease, disorder or condition is pain, neuropathic pain, epilepsy or anxiety.
